(19) United States
(12) Patent Application Publication
(10) Pub. No.: US 2022/0296390 A1
(43) Pub. Date: Sep. 22, 2022

(54) SYSTEM AND METHOD FOR DYNAMIC HIP AND SHOULDER JOINT BALANCING USING FUNCTIONAL STABILITY MEASUREMENTS

(71) Applicant: Medfit International AG, Kapfenberg (AT)

(72) Inventors: Thomas Paszicsnyek, Bruck an der Mur (AT); Christian Feigele, Bruck an der Mur (AT)

(21) Appl. No.: 17/695,922

(22) Filed: Mar. 16, 2022

Related U.S. Application Data
(60) Provisional application No. 63/161,718, filed on Mar. 16, 2021.

(51) Int. Cl. A61F 2/46

(52) U.S. Cl. CPC A61F 2/4657; A61F 2/4607; A61F 2/4609; A61F 2002/4633; A61F 2002/4666; A61B 17/15

(57) ABSTRACT

A system for orthopedic surgery comprising a client device and a plurality of position sensor units configured to communicate position information wirelessly to the client device. Each of the position sensor units comprising an anchoring means for attaching position sensor units to bone. The system further comprising an adjustable cutting block comprising an attachment portion having a recess therein for attaching the cutting block to a first of the plurality of sensor units, a cutting block portion having a second recess for attaching the cutting block to a second of the plurality of sensor units and an aperture extending through the cutting block portion for guiding a bone cutting instrument, and an intermediate portion coupling the attachment portion and the cutting block portion to each other, wherein the cutting block is therewith configured to adjustably set an orientation of the cutting instrument.

### CROSS REFERENCE TO RELATED APPLICATION

This application claims the priority of U.S. Provisional Application No. 63/161,718, entitled “SYSTEM AND METHOD FOR DYNAMIC HIP AND SHOULDER JOINT BALANCING,” filed on Mar. 16, 2021, the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to tools for assisting surgery, and in particular, sensor tools and methods for determining tension, pressure and distance in positioning components for arthroplasty surgery.

### Description of the Related Art

Hip replacement surgery has become increasingly common in the United States. During hip replacement, a surgeon removes the damaged sections of the hip joint and replaces them with an artificial hip joint usually constructed of metal, ceramic and hard plastics. The artificial hip joint typically includes a pelvic implant (cup) received in the acetabulum of the pelvis and a femoral implant (ball) at an end of a longitudinal implant portion, or a femoral implant secured to a resurfaced femoral head. One persistent issue with hip replacement is the relatively high incidence of poor placement of the cup and ball components of the prosthetic hip joint. That is, an unacceptably high percentage of patients have the cup of the artificial hip joint out of alignment with a plane of the acetabulum rim.

Different output values are of concern in hip replacement surgery. In order to reproduce a natural and/or improved gait and range of motion to a patient, the position and orientation of the implants must be considered during surgery. The work of the surgeon during hip replacement surgery will have a direct effect on these output values, and a successful surgery will relieve pain, provide motion with stability and correct deformities. The ball and socket joint of the hip needs the best possible positioning of the components to secure satisfying results. Several existing systems are available in the market available but all have their disadvantages.

Similarly, a known problem with shoulder replacement prostheses is that the positioning of the muscles and tendons in the replacement shoulder do not replicate a natural anatomical configuration. Functional stability of the shoulder is based on the relationship of the bony parts of the shoulder joint, capsule and ligaments, and the activity of the muscle envelope. Together, these provide the proper functionality of the shoulder joint along with sufficient stability. Existing shoulder replacements are more concerned with bone structure surface replacement but ignore surrounding soft tissue envelope dynamics which is crucial for the ideal functionality of the replaced joint.

Existing surgical systems and methods do not adequately help surgeons with positioning of prostheses or determine suitable stability from proper soft tissue conditions. Thus, there is a need for an arthroplasty positioning system that can aid in prostheses placement respecting muscle activity as well as the kinematic aspect of the join to achieve the best results in restoring function of joints with artificial joint replacements.

### SUMMARY OF THE INVENTION

The present invention provides systems for orthopedic surgery. According to one embodiment, the system comprises a client device and a plurality of position sensor units configured to communicate position information wirelessly to the client device. Each of the position sensor units comprising an anchoring means for attaching position sensor units to bone. The system further comprises an adjustable cutting block comprising an attachment portion having a recess therein for attaching the cutting block to a first of the plurality of sensor units, a cutting block portion having a second recess for attaching the cutting block to a second of the plurality of sensor units and an aperture extending through the cutting block portion for guiding a bone cutting instrument, and an intermediate portion coupling the attachment portion and the cutting block portion to each other, wherein the cutting block is therewith configured to adjustably set an orientation of the cutting instrument.

The client device may be configured to detect position information of each of the plurality of the sensors and construct a virtual model of a subject's skeletal anatomy based on kinematic data derived from position information from the plurality of sensors attached thereto. In one embodiment, the client device may be further configured to calculate at least one of a femoral cutting plane based on individual kinematic data and muscular activity. In a further embodiment, the client device may be configured to display an interface screen depicting an avatar of the subject skeletal anatomy and superimpose the calculated cutting plane thereon.

According to one embodiment, the system comprises a client device, a sensor cup that measures load distribution on a hip joint and transmits data including the load distribution to the client device, and a sensor prosthesis including a sensor head and an electronic connector, wherein the sensor prosthesis transmits position data to the client device.

The sensor cup may comprise a plurality of sections including pressure sensors that measure the load distribution. The client device may be configured to receive data from the sensor cup, and determine overloading, edge loading, or inadequate load distribution based on the data. The client device may be further configured to render a virtual representation of the plurality of sections of the sensor cup including color indicators that indicate right or wrong pressures in the plurality of sections. In one embodiment, the sensor head includes a spring coil and a piezoelectric actuator that lock the sensor head in a given position on a trial stem. The electronic connector may include an inner ball and an outer cup. The sensor prosthesis may be assembled by placing the sensor head in the outer cup.

In another embodiment, the client device may be configured to receive the position data from the sensor prosthesis, receive position information from a plurality of position sensor units that are attached to bones at a plurality of body positions, and determine correct positioning of the sensor prosthesis based on the position data and the position information from the plurality of position sensor units. The client device may be configured to receive the position data from the sensor prosthesis, and generate a three-dimensional model of a joint including measurements based on the position data and the position information from the plurality of position sensor units, wherein the three-dimensional model assists in making rotational and longitudinal adjustments to the sensor prosthesis.

According to one embodiment, the system comprises a client device and a tensioning device that is operated by the client device to measure a distance for shoulder prosthesis according to a given tension. The tensioning device includes a baseplate, a plurality of piezoelectric actuators embedded on the baseplate, and a hemisphere that is configured above the plurality of piezoelectric actuators.

The tensioning device may further include a wireless electronic module and a radio frequency identification chip that communicate position data to the client device. The piezoelectric actuators may be controllable by the client device to move the hemisphere in relation to the baseplate. The piezoelectric actuators may comprise peg structures that operate with spring coils. In another embodiment, the piezoelectric actuators may comprise platform structures that operate with spring coils. The piezoelectric actuators may be configured to operate either independently or collectively.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates a computing system according to an embodiment of the present invention.

FIGS. 2 through 4 illustrate a three-dimensional adjustable cutting block according to an embodiment of the present invention.

FIGS. 5 through 6 illustrates placement of a three-dimensional adjustable cutting block according to an embodiment of the present invention.

FIG. 7 illustrates an exemplary rendering of a three-dimensional model according to an embodiment of the present invention.

FIG. 8 illustrates an exemplary cutting plane according to an embodiment of the present invention.

FIGS. 9A, 9B, 9C, and 9D illustrate insertion of exemplary sensor cup according to an embodiment of the present invention.

FIGS. 10 and 11 illustrate insertion of an exemplary trial stem according to an embodiment of the present invention.

FIGS. 12A, 12B, 12C, and 12D illustrate an exemplary dynamic hip balancing system according to an embodiment of the present invention.

FIGS. 13A, 13B, 13C, 13D, and 13E illustrate an exemplary dynamic shoulder balancing system according to an embodiment of the present invention.

FIGS. 14A and 14B illustrate another exemplary dynamic shoulder balancing system according to an embodiment of the present invention.

FIGS. 15 and 16 illustrate exemplary preparation of bone surface for a tensioning device in anatomical total shoulder arthroplasty according to an embodiment of the present invention.

FIGS. 17A and 17B illustrate exemplary preparation of bone surface for a tensioning device in inverse shoulder replacement according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

The present application discloses a preferably imageless three-dimensional orientation system (as discussed in commonly-owned U.S. patent application Ser. No. 16/904,823, entitled “THREE-DIMENSIONAL ORIENTATION SYSTEM AND METHOD FOR ORTHOPEDIC SURGERY,” filed on Jun. 18, 2021, which is herein incorporated by reference in its entirety) that determines and guides users with respect to correct implant positioning using individual patient kinematics data. The orientation system may include a plurality of wireless communicating single- or multi-use sensor units that when configured as disclosed herein may be able to replace conventional instruments. Arthroplasty surgery may be performed with the sensor units in conjunction with a three-dimensional orientation system adjustable cutting block, as also disclosed herein, and trial components that are specially designed to operate with the orientation system. As such, the presently disclosed system(s) enable performing arthroplasty on, e.g., ball and socket joints, such as the hip or shoulder, that account for individual kinematics, kinetics, abilities and/or muscle status, which may be retrieved from a muscle activity controlled physiotherapy (“MacP”) system that measures and examines muscular ability (as discussed in commonly-owned U.S. patent application Ser. No. 17/482,028, entitled “SYSTEM AND METHOD FOR AIDING IN PHYSIOTHERAPY BY MEASURING MUSCLE ACTIVITY USING WIRELESS SENSORS,” filed on Sep. 22, 2021, which is incorporated herein by reference). Although the systems may be described herein in relation to arthroplasty with respect to ball and socket joints, it is understood that the systems are equally useful for other joints and non-human subjects, and are therefore not limited thereto.

The disclosed systems preferably include a plurality of positioning sensors which can be placed on pre-defined anatomical landmarks in a body area to be operated on, such as a ball and socket joint, and one or more plane-marker sensors for fixing an adjustable cutting block relative to the joint bone being cut, preferably adjustable in three dimensions, in the desired position. The sensors may be optical, preferably non-optical, or a combination thereof. Optical sensors may include markers that are observed by system cameras, for example, to determine the position of the markers in a three dimensional space. Non-optical systems may include inertial units (referred to herein generally as “gyro” units), which may include at least one of a gyroscope, magnetometer, accelerometer, radio frequency identification (“RFID”) chip, or a combination thereof, to measure the position and/or orientation of each of the sensor units in three dimensions. Similarly, magnetic systems may be used to calculate position and/or orientation of the sensor unit based on the relative magnetic flux of the sensor units to three orthogonal coils. Together with an interface and a software application, the system may calculate the orientation of cutting planes, enabling a proper physiological configuration based on individual patient kinematic data. The relation between the individual sensors together with their position in a three-dimensional space results in, e.g., a three-dimensional, trajectory system. Muscular data may be acquired to perform gait analysis and load distribution analysis for configuring optimal post-operative treatment protocols in accordance with individual capabilities and muscle function, which is discussed in further detail in commonly-owned U.S. Patent Application No. 63/176,079, entitled “SYSTEM AND METHOD FOR FUNCTIONAL STABILITY PLANNING OF REPLACEMENT JOINTS,” filed on Apr. 16, 2021, which is incorporated herein by reference.

According to one embodiment, the disclosed system further comprises a dynamic hip balancing device including active sensors that transmit data to a computing device executing a dedicated application that calculates and determines positioning for hip prosthesis placement. The disclosed system may identify an individual kinematic situation of a specific patient including the pelvic motion and range of motion as well as individual limitations and abilities. Further examination of the muscular ability measurement from a MacP system can give additional information to the individual condition of the patient and can affect the ideal position of cup and stem components of the hip replacement. Based on data gathered preoperatively by the MacP system, or functional stability planning and initial X-rays, or interoperative data including functional measurement results, an ideal cup position can be determined.

After performing a first cut at the femoral neck, inserting a sensor trial cup in a reamed diameter of the acetabulum, and placing a trial stem in the intramedullary space, a sensor device may be mounted to the stem to perform an initial measurement. Also by mounting the sensor device on a hip cup impactor (handheld device used during hip joint replacement surgeries to implant cups in the acetabulum), the correct plane for the sensor trial cup can be attained. A first measurement may be performed to determine load distribution in a cup component to verify edge loading or loosening that may cause dislocation. The load distribution may be measured for an entire or given range of motion. The first measurements can be used to determine correct positions of the cup component and a stem which can be set and stored to the dedicated application. A selected implant (cup and stem) can then be implanted and with a tensioning device, a control measurement for stability may be performed. The control measurement can be performed with the disclosed system to choose the right length of the femoral head. If done with the trial stem, the stem position can also be determined by the disclosed system. Separately the range-of-motion, offset position, and functional movement can be checked with the selected implant using the plurality of positioning sensors. As such, functional stability of the joint may be compared before and after implanting the prosthesis.

According to another embodiment, the disclosed system further comprises a dynamic shoulder balancing including an active tensioner device that is wirelessly connected to a computing device executing a dedicated application that determines the correct tension and replacement position of shoulder prosthesis. The dynamic shoulder balancing device may be comprised of biocompatible plastic with embedded electronic parts for tensioning, positioning, and wireless communications (e.g., via a Bluetooth connection). The dynamic shoulder balancing device may be intended for single use to indicate correct implant height and dimensions, and tension of a soft tissue envelope (e.g., ligaments, muscle, and capsule).

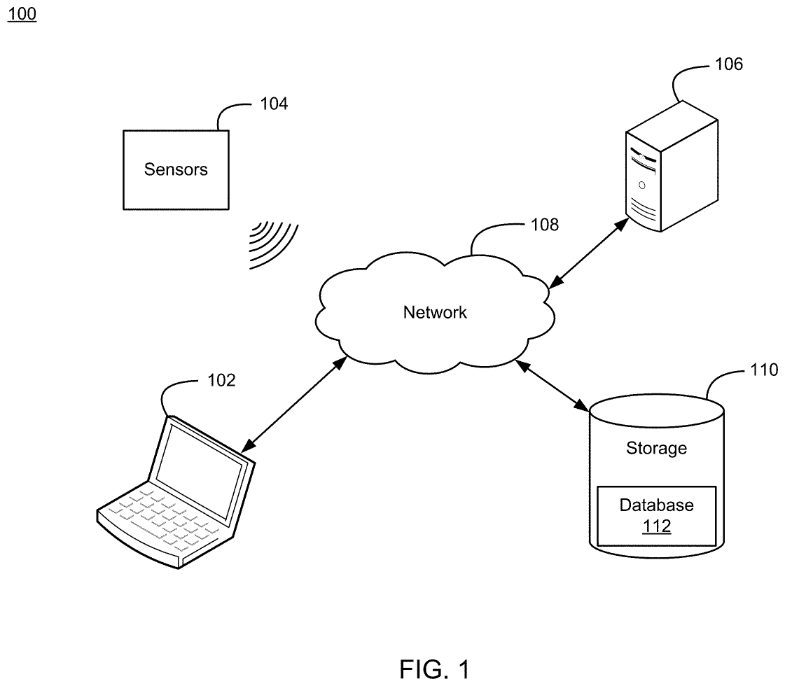

FIG. 1 illustrates a computing system according to an embodiment of the present invention. The system presented in FIG. 1, in accordance with at least one embodiment of the systems disclosed herein, includes one or more client device 102, a plurality of sensors 104, one or more servers 106, and at least partially wireless network 108, and storage 110. Depending on the type of sensor, the system may further include a receiver, such as a camera or magnetometer, which communicates position information obtained from or with the sensors 104 to the other systems components, over the network 108 or otherwise, as discussed herein. The receiver, e.g., a camera, may further detect gestures by users for hands free input by the surgeon, as explained below.

Client device 102 may include computing devices (e.g., desktop computers, terminals, laptops, personal digital assistants (PDA), cellular phones, smartphones, tablet computers, or any computing device having a central processing unit and memory unit capable of connecting to a network). The client device 102 may also include a graphical user interface (GUI) or a browser application provided on a display (e.g., monitor screen, LCD or LED display, projector, etc.). Client device 102 may also include or execute an application to communicate content, such as, for example, textual content, multimedia content, or the like, including content provided with the interface screens disclosed herein. The client device 102 may also include or execute an application or app to perform a variety of possible tasks, such as browsing, searching, playing various forms of content, including locally stored or streamed video, data input and output, etc. Client device 102 may include or execute a variety of operating systems, including a personal computer operating system, such as a Windows, Mac OS or Linux, or a mobile operating system, such as iOS, Android, or Windows Phone, or the like. Client device 102 may also include or may execute a variety of possible applications, such as a client software application enabling communication with other devices or components, such as communicating one or more messages, such as via email, short message service (SMS), or multimedia message service (MMS), including via a network.

Sensors 104 according to at least one embodiment include gyro sensor units that each include movement tracking, power, circuitry, and wireless communication components. The gyro sensors 104 may detect rotational motion and changes in orientation, and communicate that information to the system 100 for tracking the 3D position of the sensors 104 in three dimensions. The sensors 104 may be for single-use only and each have a defined size and shape to fit certain dimensions (e.g., 20×20×8 mm). The sensors 104 preferably include pins or other anchoring devices for attaching the individual sensor to the bones of the body part being operated on and individually activated to send active signals to client device 102 either over network 108 or directly via short-range wireless communication (e.g., Bluetooth, Near-Field Communication, etc.). Each of sensors 104 may be activated by a magnetic switch, and identified or labeled according to their position relative to a plurality of predefined positions, and registered with an interface on client device 102.

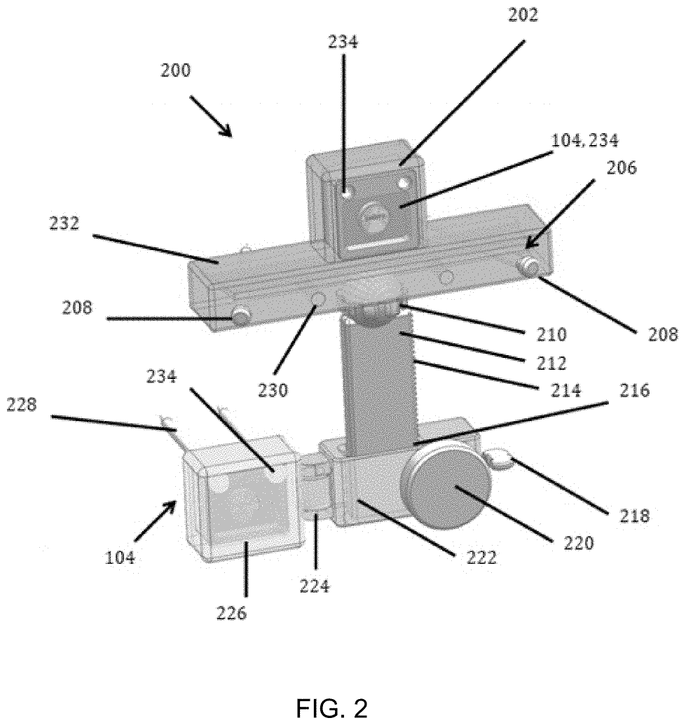

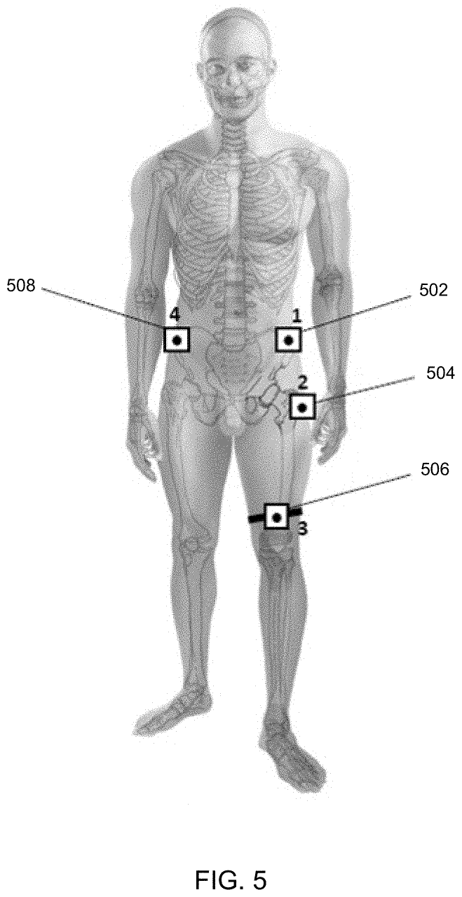

The sensors 104 in communication with client device 102 may, once activated, form a three-dimensional matrix or grid reference system such that an orientation of, for example, the acetabulum and the femoral joint surface can be modeled and validated with the system 100, as discussed further below. The system further includes a three-dimensional adjustable cutting block 200 (FIGS. 2-4), which is configured to attach to one or more of the sensors 104 previously affixed to bone, as shown in FIG. 5. The cutting block 200 includes one or more sensors 234 for determining the position and orientation of the cutting plane (e.g., plane-markers), which may be adjusted at specific relative locations to other ones of the sensors 104 to set up the desired individual orientation for cutting the femoral head. The adjustable cutting block 200 may be used for hip, shoulder, and knee using the three-dimensional orientation system for finding a correct cutting plane.

Referring to FIG. 2, the three-dimensional adjustable cutting block 200 includes cutting block portion 232, which includes a recess 202 therein for attaching thereto a plane-marker sensor 234 and an aperture 206 that sets the orientation of the plane for guiding a cutting instrument with respect to cutting bone in preparation for the implant. The aperture 206 may be a slot with opposing planer surfaces spaced apart from each other to guide an oscillating bone cutting saw blade, for example. The cutting block 200 further include holes 230 for fixing the cutting portion 232 to the bone with, for example, pins 208 through holes 230, when the cutting block 232 is adjusted to achieve the desired cutting plane orientation. The cutting block 200 further includes a first attachment portion 226 that has a recess therein for attaching the block 200 to a sensor 104, which has been affixed previously to the bones of a joint, e.g., with pins 228 passing through holes 234 in the sensor 104, as discussed herein. As discussed above, the sensors have a defined shape and size so that the cutting block 200 can be attached to any one of the sensors 104, and thus the sensors 104 can be used interchangeably prior to registration with the system.

The first attachment portion 226 and cutting block portion 232 are adjustable relative to each other, preferably in three dimensions. This may be achieved in a variety of ways. As shown in FIG. 2, articulation in this regard may be achieved with an intermediate portion that includes one or more swivel connections 210, 224, hinges 224, and/or a telescopic mechanism(s), such as rack 212 and pinion 222. The rack 212 and pinion 222 system includes an adjuster 220 with teeth 216 thereon that engage corresponding teeth 214 on rack 212 to adjustably extend or retract cutting block portion 220 telescopically, i.e., in the vertical (y) direction. This “height” may be locked into place with a locking screw 218. The adjustability of the cutting plane may be enabled with hinge 224 or/or with swivels 210, 224. In the embodiment shown in FIG. 2, the cutting plane presented by block 232 is adjustable vertically, rotationally about the vertical axis, and pivotally.

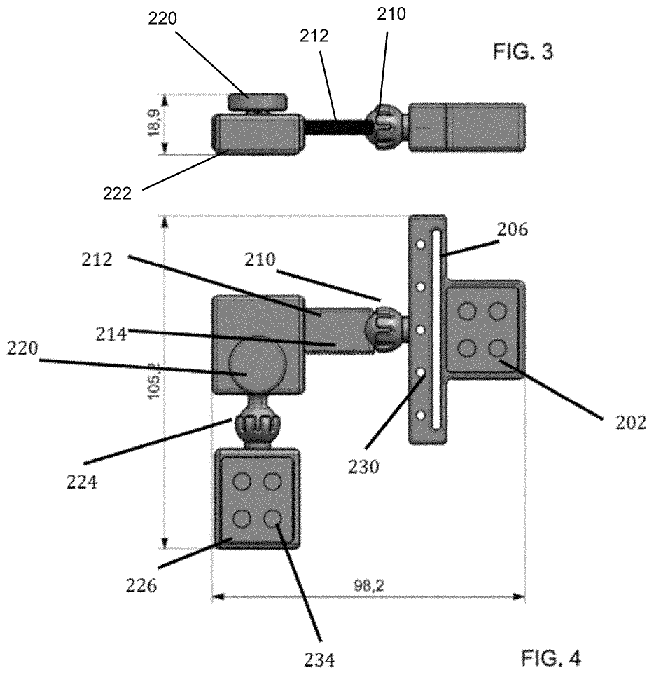

The three-dimensional adjustable cutting block 200 may have an exemplary length and width of about 98.2 mm×105.2 mm, but may be any dimension suitable for the joint at issue. A portion of the three-dimensional adjustable cutting block including the adjuster may have an exemplary thickness of about 18.9 mm. In at least one embodiment, as shown in FIGS. 3-4, the adjustable cutting block includes a first attachment portion 226 coupled to an intermediate telescopic portion, which is further coupled to the cutting block portion 232. The first attachment portion 226 and the cutting block portion 232 preferably each include the same sized compartment for receiving and attaching to at least one sensor therein. The intermediate portion is preferably coupled to the other portions with a hinge or pivot that allows the user to adjust the cutting portion relative to the first attachment portion. The intermediate portion preferably includes a pair of structure operable to orthogonally slide relative to each other, which allows the user to adjust the distance between the sensors, and correspondingly the distance of the cutting plane relative to the sensor to which the cutting block 200 is attached.

Server 106 may include a computing system operable to allow access to storage 110 over network 108. Network 108 may be any suitable type of network allowing transport of data communications across thereof. The network 108 may couple devices so that communications may be exchanged, such as between server 106 and client device 102 or other types of devices, including between wireless devices coupled via a wireless network, for example. The network 108 may also include mass storage, such as network attached storage (NAS), a storage area network (SAN), cloud computing and storage, or other forms of computer or machine-readable media, for example. In one embodiment, the network 108 may be the Internet, following known Internet protocols for data communication, or any other communication network, e.g., any local area network (LAN) or wide area network (WAN) connection, cellular network, wire-line type connections, wireless type connections, or any combination thereof.

Server 106 may include at least a special-purpose digital computing device including at least one or more central processing units and memory. The server 106 may also include one or more of mass storage devices, power supplies, wired or wireless network interfaces, input/output interfaces, and operating systems, such as Windows Server, Mac OS X, Unix, Linux, FreeBSD, or the like. In an example embodiment, server 106 may include or have access to memory or computer readable storage devices for storing instructions or applications for the performance of various functions and a corresponding processor for executing stored instructions or applications. For example, the memory may store an instance of the server configured to operate in accordance with the disclosed embodiments.

Server 106 is operative to receive requests from client device 102 to retrieve analysis and procedure data of a patient including a virtual model of a given body part from database 112. Prior to surgery, a preliminary examination may be performed with optical markers and electrodes attached to wearable stockings (or any other wearable article) to identify a target patient's movement and kinematic data. This may be done with a special movement analysis with 3D cameras together with an electromyography (“EMG”) measurement to value the activity of the individual muscles of the joint along with MacP and functional stability planning of replacement joints system discussed above. While wearing the stocking, the patient may be analyzed during walking on a treadmill, climbing stairs up and down, and moving a joint against a defined resistance in flexion and extension. The analysis may be conducted under standardized conditions, such as in a specialized testing structure including stairs, treadmill, bench, and testing equipment. Accordingly, different values of muscular activity may be recorded and analyzed by either client 102 or server 106. These values can be compared to the other side (contralateral), ideally the healthy one. If the contralateral side is also affected, a template database of healthy patients (stored in database 112) may be used for comparison.

The analysis may be performed, together with long leg x-ray from the front (coronal plane) and the side (sagittal plane), to produce a three-dimensional active avatar of a whole leg or body part. The avatar may include a virtual leg or body part moving with respect to individual muscle activity and the anatomical situation of the joint capsule, ligaments and bones. Such a virtual model can be used to virtually implement the prosthesis and determine the correct three-dimensional orientation of various skeletal planes. The data generated for the avatar may be the basis for a correct cutting plane (e.g., in connection with the functional stability planning of replacement joints system) and may be stored to database 112. For example, client device 102 may generate an interface depicting an avatar of the subject's skeletal anatomy and superimpose a calculated cutting plane. The virtual model when used in conjunction with an anamnesis of the progress of disease, a surgeon can decide which protocol to follow.

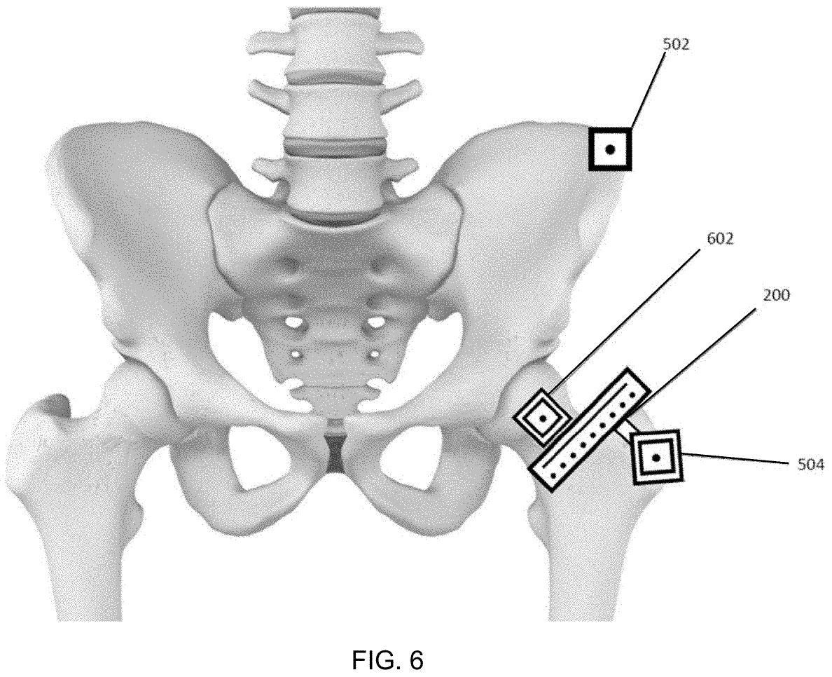

A client device 102 may be configured to communicate with sensors 104 installed in certain positions on the bone of a body part being operated on. In one example, after opening to expose a joint, ideally preserving all functional structures, especially the muscle envelope, sensors 104 may be placed on defined landmarks, e.g., on the subject's spina iliaca anterior superior 502, trochanter major 504, anterior surface of the leg 506 (near the knee joint), and at the spina iliaca anterior superior 508 on the contralateral side, as shown in FIG. 5, as well as a sensor 602 on the cutting block 200 itself (FIG. 6). These sensors 104 are preferably secured so that they do not interfere with the cutting planes established with the cutting block 200.

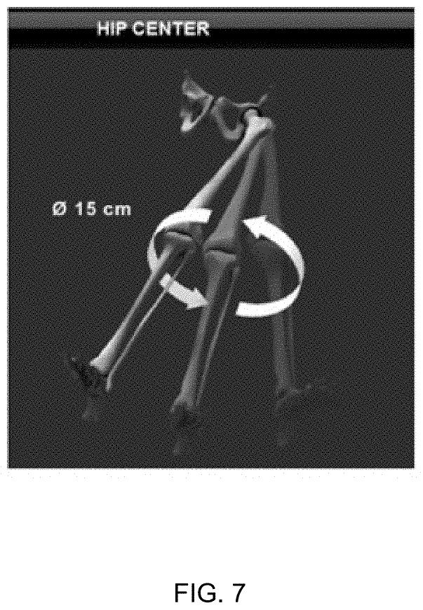

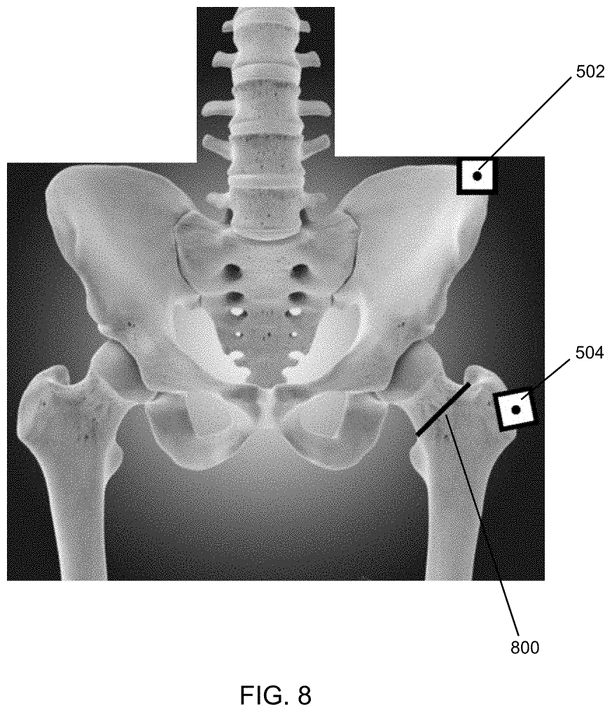

FIG. 7 presents an exemplary rendering of a three-dimensional model according to an embodiment of the present invention. The placements of the sensors 104 on the subject's skeletal anatomy generally enables the system to create a model of the skeletal anatomy in a three-dimensional reference grid or system, e.g., a 3D model, which may be generated by the client device 102 and/or server (e.g., via the three-dimensional orientation system) based on the location information obtained by the client device 102 and/or server from respective sensors 104 communicating with the client device 102 and/or server. In case of preoperative planning, the three-dimensional model may be compared with a preoperatively calculated reference model. The model generated by the system may include location information of each of the sensors 104 throughout the range of movement of the joint. In this regard, location data capture may include performing several flexion and extension as well as rotational movements of the joint, as shown in FIG. 7, until a software interface of the client device 102 shows that the system acquired the data necessary to generate the 3D model of the joint, e.g., three-dimensional positioning of the joint, through at least a portion of the range of movement of the joint. The location data may be captured as part of a calibration process that can be compared to preoperative x-rays and respective optional muscle activity measurement and gait analysis. All of the data can be used to determine the ideal plane for cutting the femoral neck, which can be performed by positioning the cutting block 200 according to the determined ideal plane (cutting plane 800 in FIG. 8).

As an option, an orientation screen may be presented or otherwise displayed by the client device 102 to guide the use in setting up the proper orientation of the three-dimensional adjustable cutting block 200 and more specifically the cutting block portion 232 thereof. Beside the orientation of the plane, the thickness of the cut may be determined by the client device 102 such that the bone loss can be minimized as much as possible. When the orientation and/or thickness of the cut is verified by the system based on information from the sensor attached to the cutting block portion 232, the three-dimensional cutting block 200 may be fixed by pins for maximum available stability during the cutting. When the surgeon has verified the plane and/or thickness of the cut is proper, the cut of the plane can finally be performed. The discussed modeling and analysis with respect to the three-dimensional orientation system and the functional stability planning of replacement joints system are optional but not required for use with the dynamic hip balancing system disclosed herewith.

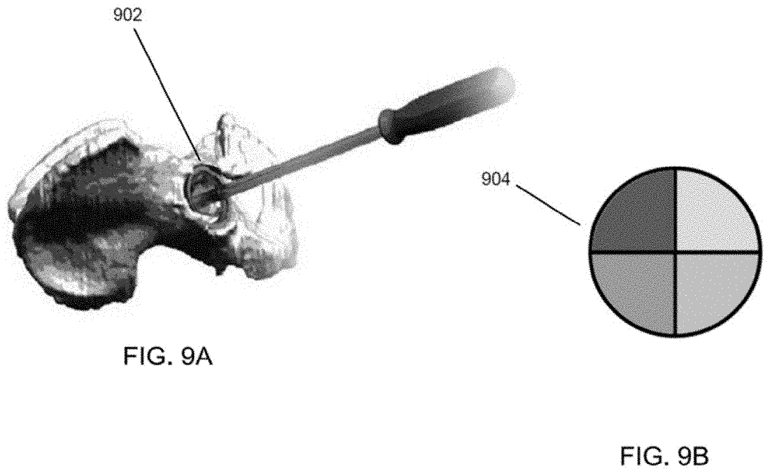

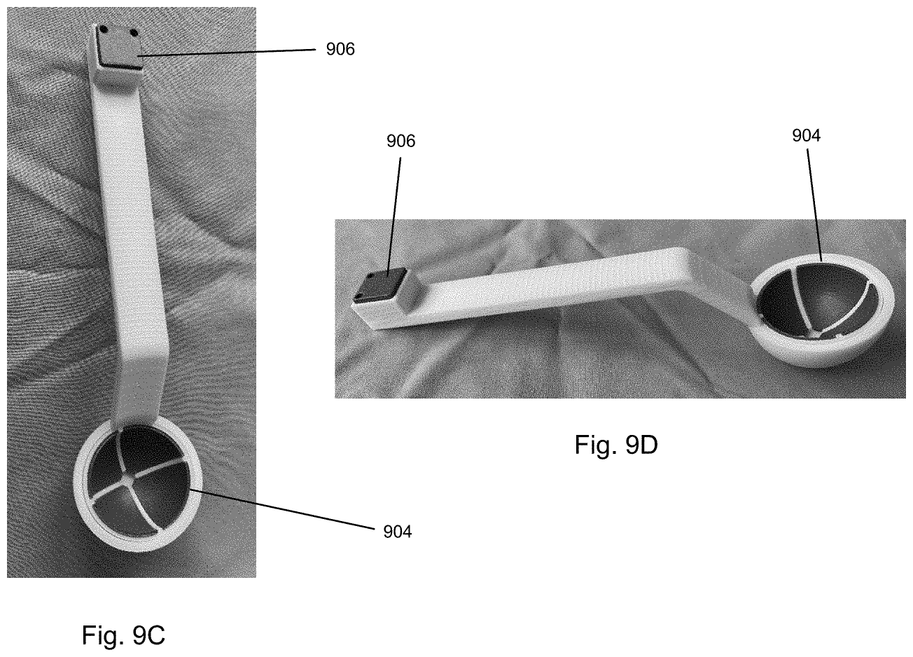

Referring to FIGS. 9A through 9D, a dynamic hip balancing system comprises a sensor cup 904 that may be used by client device 102 to determine proper load distribution on hip joint and correct positioning for ideal load distribution to increase survivorship of the implant. After performing the cut, the acetabulum 902 may be reamed to an appropriate diameter and the sensor cup 904 may be inserted into the reamed acetaulum 902. This sensor cup 904 may be placed into the reamed acetabulum 902 according to the reamed diameter (for example, reamer size 50 corresponds to a size 50 sensor cup). The sizes may be anywhere within the range from 42 mm to 62 mm in 2 mm steps.

FIGS. 9C and 9D illustrate views of the sensor cup 904 including a handgrip with a sensor 906 for communicating positioning and alignment data of the sensor cup 904 when placing in the reamed acetabulum 902. The surface of sensor cup 904 is divided into a plurality of sections or quadrants. Each quadrant may include pressure sensors that measure the exact load distribution and transmit data to client 102 which can interpret the data and warn in case of overloading, edge loading, or inadequate load distribution. For example, the computer may generate a visual interface that renders a virtual representation of the sensor cup quadrants including color indicators for each of the quadrants to indicate right or wrong pressures in each respective quadrant of the sensor cup 904. Green may indicate an appropriate pressure, while yellow may indicate a certain degree of imperfect pressure, and red may indicate an incorrect pressure. The appropriate amount of pressure may be determined based on a database of healthy patients, e.g., database 112, including data of a range of muscle strength for each joint by age group.

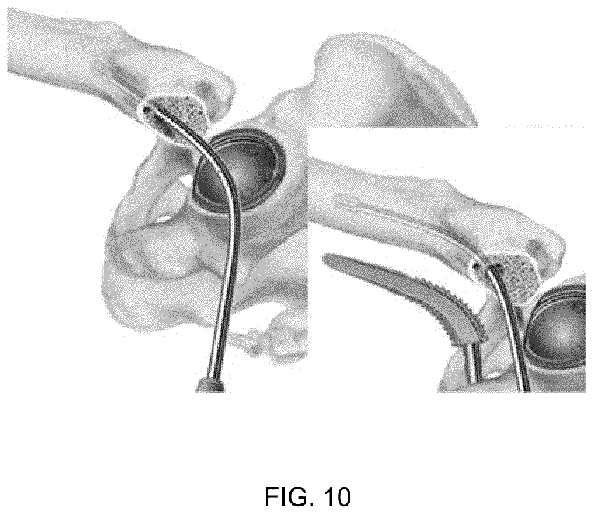

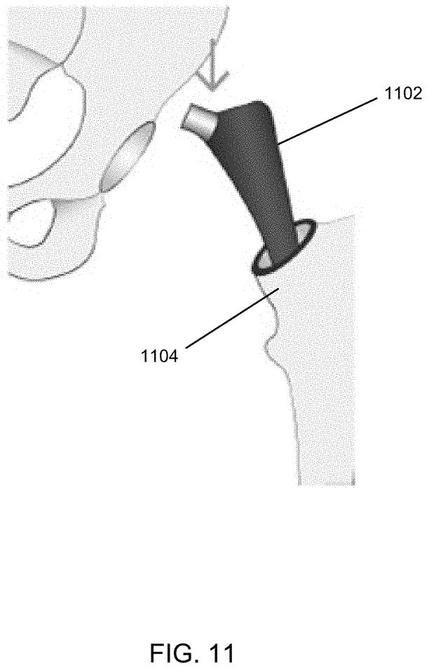

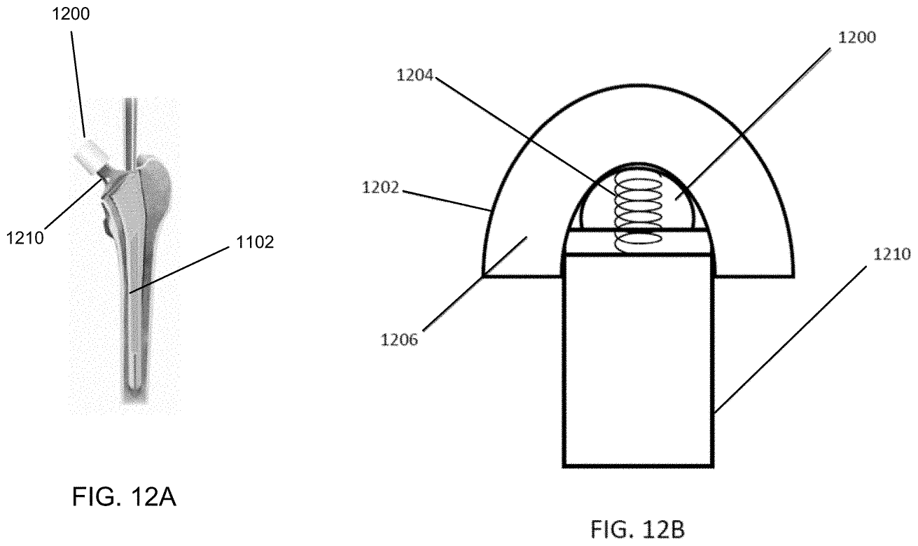

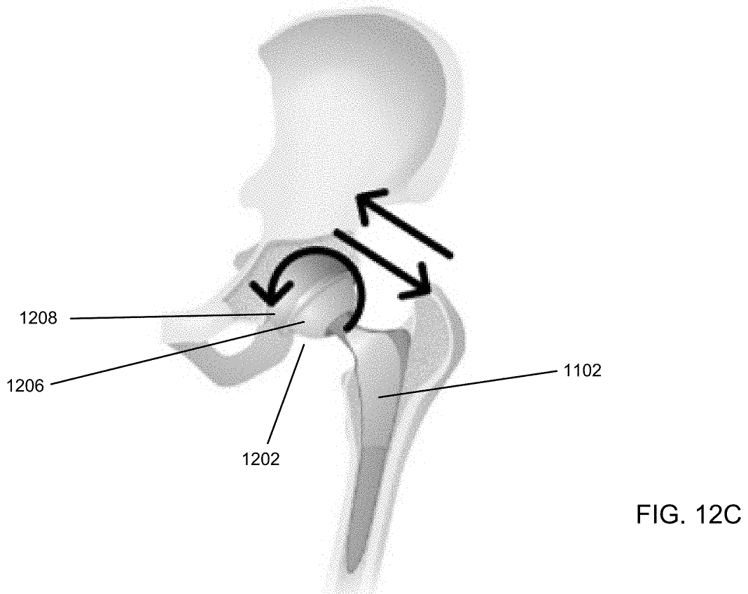

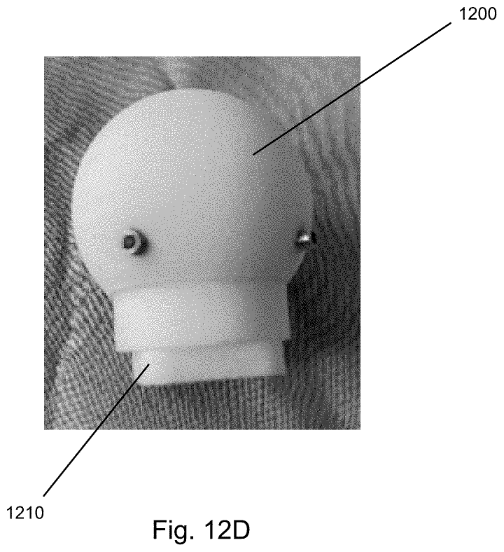

A femoral diaphysis may be rasped (FIG. 10) to an appropriate stem size such that a trial stem 1102 can be placed in the femur 1104, as illustrated in FIG. 11. A sensor prosthesis may be used on the trial stem 1102 to determine a proper size and position of a selected prosthesis. Referring to FIGS. 12A, 12B, 12C, and 12D a sensor prosthesis comprises a sensor head 1200 and an electronic connector 1202. Inside the sensor head 1200 includes a spring coil 1204 and a piezoelectric actuator (not illustrated).

The sensor head 1200 may be compressed and released for adjusting correct tension when placed in the femur. The spring coil 1204 may be configured to tension soft tissue according to an optimal soft tissue parameter. The sensor head 1200 may be temporarily locked in an appropriate position for measurement and such that the length of the neck 1210 provides proper load distribution. The sensor head 1200 may be released to be removed. The electronic connector 1202 includes an inner ball 1206 and an outer cup 1208. The electronic connector 1202 may be compatible with conventional diameters of a neck and a head (e.g., 28 mm to 40 mm diameter) of a total hip arthroplasty implant. The sensor prosthesis may be assembled by placing sensor head 1200 in the outer cup 1208 according to the implant diameter (e.g., 28 mm, 32 mm, 36 mm and 40 mm).

The sensor head 1000 may be placed on the trial stem 1102 with the spring coil 1204 released. FIG. 12C shows possible adjustments of the position of the cup as well as the tension status of the head. Upon reaching a predefined position, the sensor head 1200 is allowed to be locked by the piezoelectric actuator to perform a measurement to compare with pre-operative measurements and planned measurements.

The sensor prosthesis may send position data to an application executing on client device 102 which may receive the position data along with data from sensors 104 and sensor cup 904 to determine correct positioning of the sensor prosthesis. The application determine correct positioning of the final prosthesis for the replacement procedure based on three-dimensional measurements created from the received data in combination with pre-operative data. The application may generate, for example, a three-dimensional model of the joint on a user interface including measurements based on all the data received by the application (e.g., from sensor prosthesis, sensors 104, and sensor cup 904) and assist or guide a surgeon in making rotational and longitudinal adjustments to the sensor prosthesis. Leg length and offset of the leg may be compared by the application to pre-operative data such that the surgeon will be able to correct any kind of mispositioning. The ante- and retroversion of the stem can also be determined with data from the dynamic hip balancing device and the sensors 104 and sensor cup 904. Once the correct position of the cup and stem as well as the length and offset are determined, the correct sizes of all implants may be determined with correct three-dimensional positioning by the application. As such, the surgeon may be able to adapt the position of cup, stem, and head components of a total hip arthroplasty implant according to a patient's specific anatomy using sensor measurements gathered by the application. After placing the final prosthesis, a final control measurement can be performed to gather post-operative data that may be stored in the database. Post-operative x-rays of the patient may also be compared to pre-operative x-rays and planned/expected prosthesis positioning for quality control.

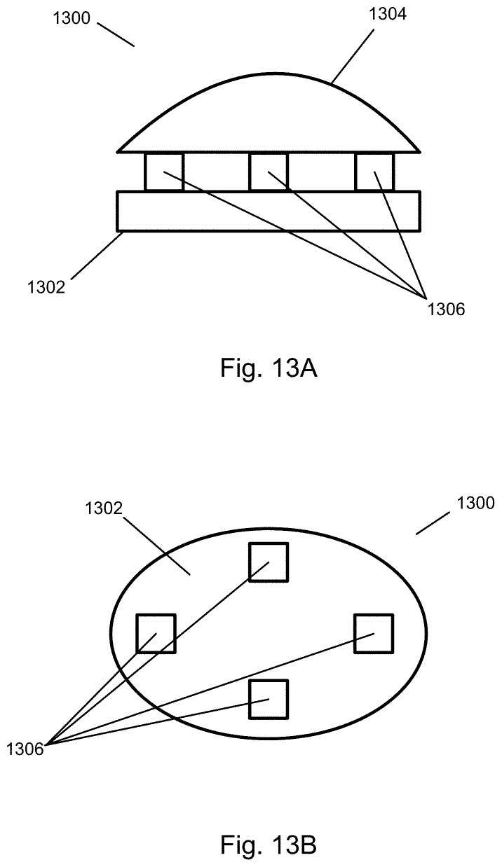

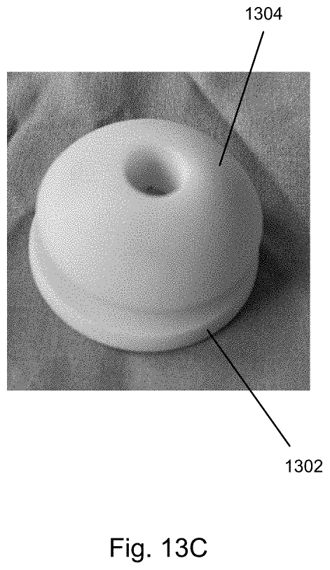

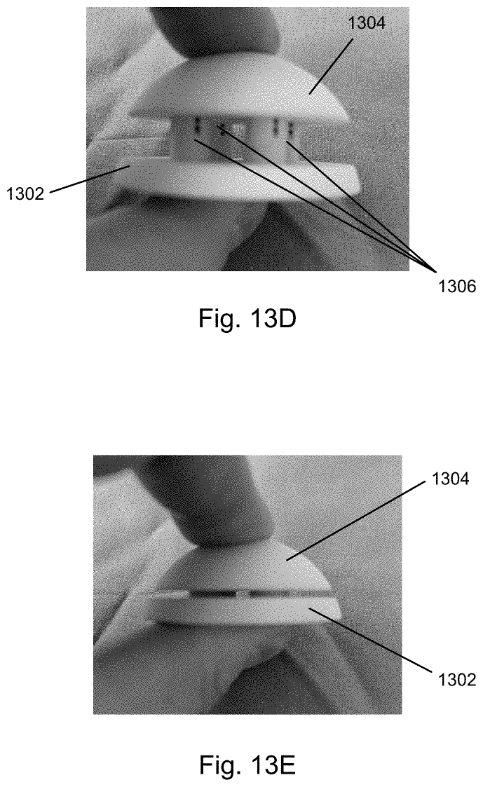

FIGS. 13A through 13E present a dynamic shoulder balancing system according to an embodiment of the present invention. The dynamic shoulder balancing system comprises a tensioning device 1300 including baseplate 1302, hemisphere 1304 and piezoelectric actuators 1306. The tensioning device 1300 further includes a wireless electronic module (e.g., Bluetooth) for connection to a client device 102 and a RFID chip for position data acquisition by the client device 102. Piezoelectric actuators 1306 is embedded on or affixed to baseplate 1302. The hemisphere 1304 is configured on top of the piezoelectric actuators 1306 such that baseplate 1302 may remain stationary while hemisphere 1304 is movable in relation to the baseplate 1302 by piezoelectric actuators 1306 which can be controlled by the client device 102. After a calibration process with the client device 102, the tensioning device 1300 may be situated on a bone, either the humerus for anatomical total shoulder arthroplasty or on the glenoid for inverse total shoulder arthroplasty.

The tensioning device 1300 may be activated by the client device 102 to control the piezoelectric actuators 1306. The piezoelectric actuators 1306 include peg structures that operate with spring coils (not illustrated). The tensioning device 1300 may tension up the soft tissue envelope surrounding the bone to a defined tension by lifting and lowering the hemisphere 1304 with the piezoelectric actuators 1306. Each of the piezoelectric actuators 1306 may be moved or operated either independently or collectively. Additionally, hemisphere 1304 may be compressed and released. The client device 102 may obtain and tune the distance between the baseplate 1302 and hemisphere 1304 by adjusting the piezoelectric actuators 1306. After reaching a correct or desired position, the actuators 1306 may be locked for checking and released for testing.

The distance between the baseplate 1302 and hemisphere 1304 may be set based on an appropriate tension value (e.g., 6 mm at 20 newtons) that may be measured by tensioning device 1300 and transmitted to the client device 102. Particularly, distance values at each of the piezoelectric actuators 1306 may be used as a reference for implantation of a shoulder prosthesis. The piezoelectric actuators 1306 further include sensors that may record and transmit measurement data to client device 102. The measurement data may be used by the client device 102 to generate a display of measurements in quadrants corresponding to the piezoelectric actuators 1306. The measurements data may also be used by the client device 102 in combination with data from sensors 104 to generate a three-dimensional joint movement model (e.g., via the three-dimensional orientation system and the functional stability planning of replacement joints system) that shows prosthesis positioning and any deviation which may be outside a tolerable range.

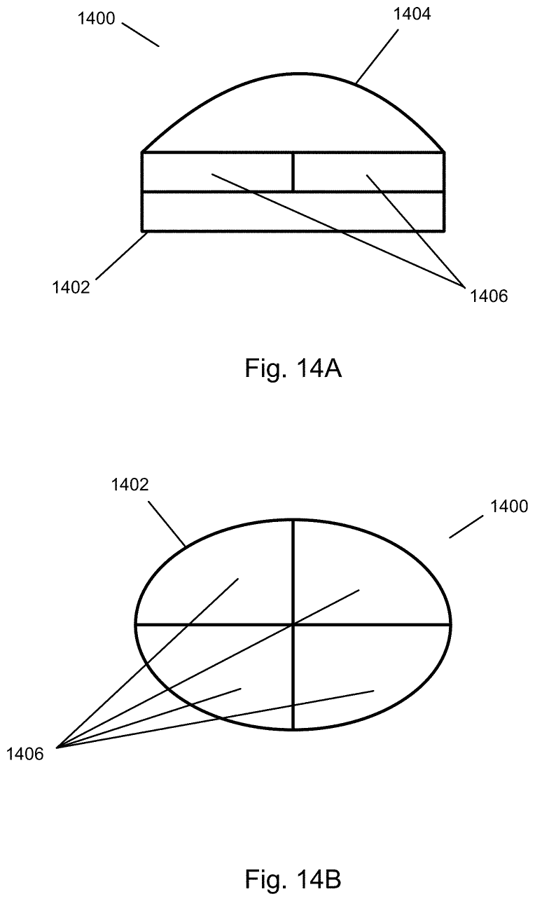

FIGS. 14A and 14B present another exemplary dynamic shoulder balancing system according to an embodiment of the present invention. The dynamic shoulder balancing system comprises a tensioning device 1400 including baseplate 1402, hemisphere 1404 and piezoelectric actuators 1406. The tensioning device 1400 further includes a wireless electronic module (e.g., Bluetooth) for connection to a client device 102 and a radio frequency identification (“RFID”) chip for position data acquisition by the client device 102. Piezoelectric actuators 1406 is embedded on or affixed to baseplate 1402. The hemisphere 1404 is configured on top of the piezoelectric actuators 1406 such that baseplate 1402 may remain stationary while hemisphere 1404 is movable in relation to the baseplate 1402 by piezoelectric actuators 1406 which can be controlled by the client device 102. After a calibration process with the client device 102, the tensioning device 1400 may be situated on a bone, either the humerus for anatomical total shoulder arthroplasty or on the glenoid for inverse total shoulder arthroplasty.

The tensioning device 1400 may be activated by the client device 102 to control the piezoelectric actuators 1406. The piezoelectric actuators 1406 include platform structures that operate with spring coils (not illustrated). The tensioning device 1400 may tension up the soft tissue envelope surrounding the bone to a defined tension by lifting and lowering the hemisphere 1404 with the piezoelectric actuators 1406. Each of the piezoelectric actuators 1406 may be moved or operated either independently or collectively. The client device 102 may obtain and tune the distance between the baseplate 1402 and hemisphere 1404 by adjusting the piezoelectric actuators 1406.

The distance between the baseplate 1402 and hemisphere 1404 may be set based on an appropriate tension value (e.g., 6 mm at 20 newtons) that may be measured by tensioning device 1400 and transmitted to the client device 102. Particularly, distance values at each of the piezoelectric actuators 1406 may be used as a reference for implantation of a shoulder prosthesis. The piezoelectric actuators 1406 further include sensors that may record and transmit measurement data to client device 102. The measurement data may be used by the client device 102 to generate a display of measurements in quadrants corresponding to the piezoelectric actuators 1406. The measurements data may also be used by the client device 102 in combination with data from sensors 104 to generate a three-dimensional joint movement model that shows prosthesis positioning and any deviation which may be outside a tolerable range.

The tensioning devices according to the embodiments discussed above may be used to obtain reference measurements after bone cutting. It is noted that either one of tensioning device 1300 or tensioning device 1400 may be used for both anatomical total shoulder arthroplasty and inverse total shoulder replacement.

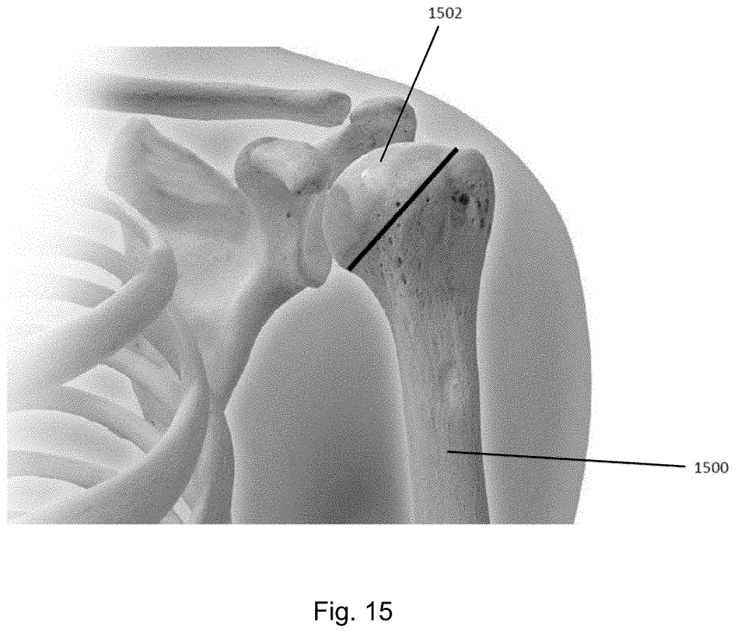

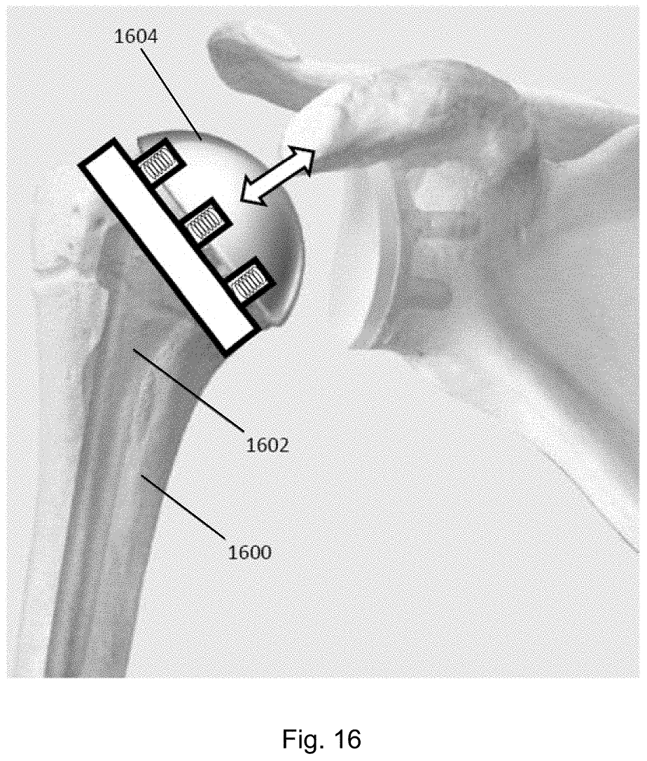

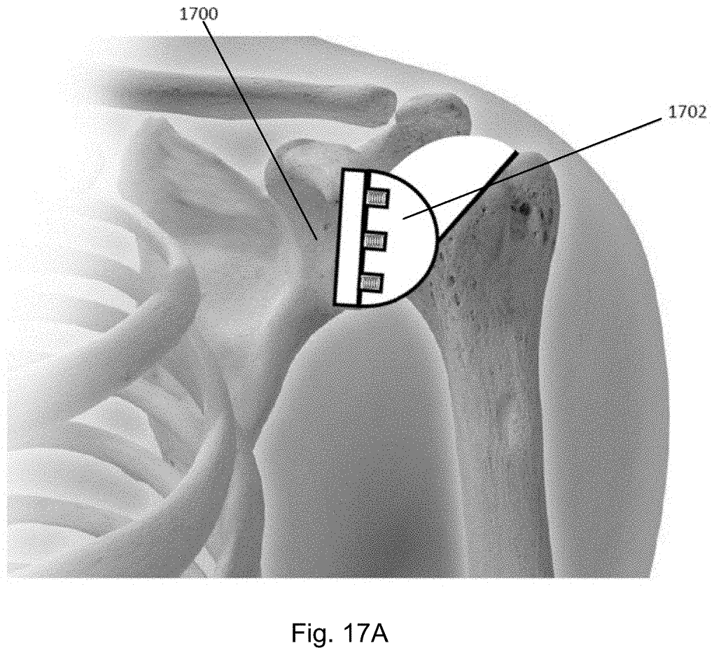

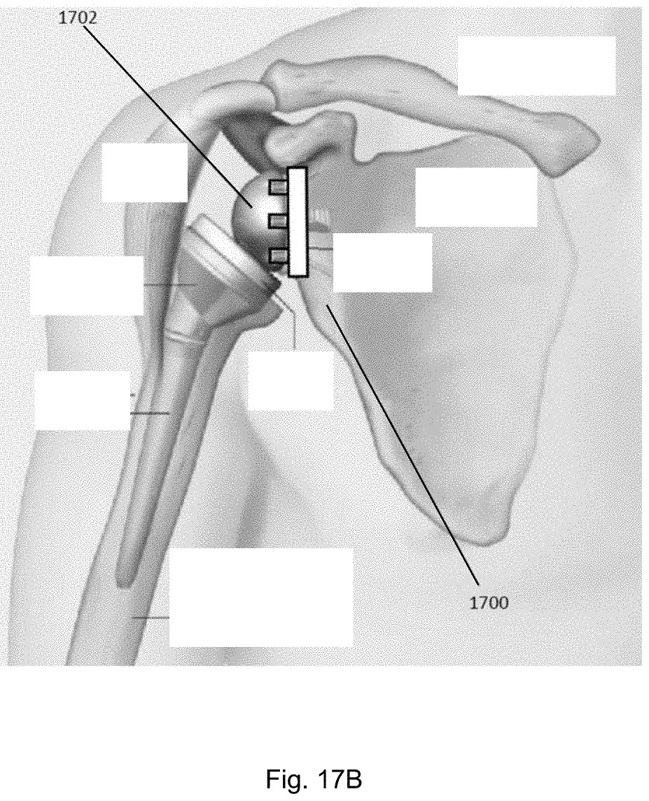

A portion 1502 of the humerus 1500 is resected for anatomical total shoulder arthroplasty, as illustrated in FIG. 15. Referring to FIG. 16, anatomical total shoulder arthroplasty further includes rasping a resected humerus 1600 for insertion of a stem 1602. A tensioning device 1604 is connected to the stem 1602 by a compatible adapter (not illustrated) and is used to obtain another set of measurements for comparison with the reference measurements. Similarly, a tensioning device 1702 is attached to glenoid 1700 for inverse total shoulder replacement as shown in FIGS. 17A and 17B. Tensioning device 1702 may comprise components that are similar or identical to tensioning device 1604.

If a correct or stable balance is achieved based on the measurements, the tensioning device may be removed from the stem 1602 or glenoid 1700 and replaced with a selected shoulder prosthesis (e.g., a glenosphere). Post-operative measurements can be performed and stored to a database where it may be compared to pre-operative data and planned/expected prosthesis positioning for quality control.

FIGS. 1 through 17B are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A system for orthopedic surgery, comprising:
a client device;
a plurality of position sensor units configured to communicate position information wirelessly to the client device, wherein each of the position sensor units comprises an anchoring means for attaching position sensor units to bone; and
an adjustable cutting block comprising:
an attachment portion having a recess therein for attaching the cutting block to a first of the plurality of sensor units;
a cutting block portion having a second recess for attaching the cutting block to a second of the plurality of sensor units and an aperture extending through the cutting block portion for guiding a bone cutting instrument; and
an intermediate portion coupling the attachment portion and the cutting block portion to each other, the cutting block therewith configured to adjustably set an orientation of the cutting instrument.

2. The system of claim 1, wherein the client device is configured to detect position information of each of the plurality of the sensors and construct a virtual model of a subject's skeletal anatomy based on kinematic data derived from position information from the plurality of sensors attached thereto.

3. The system of claim 2, wherein the client device is further configured to calculate at least one of a femoral cutting plane based on individual kinematic data and muscular activity.

4. The system of claim 3, wherein the client device is further configured to display an interface screen depicting an avatar of the subject skeletal anatomy and superimpose the calculated cutting plane thereon.

5. A dynamic hip balancing system comprising:
a client device;
a sensor cup that measures load distribution on a hip joint and transmits data including the load distribution to the client device; and
a sensor prosthesis including a sensor head and an electronic connector, the sensor prosthesis transmits position data to the client device.

6. The dynamic hip balancing system of claim 5 wherein the sensor cup comprises a plurality of sections including pressure sensors that measure the load distribution.

7. The dynamic hip balancing system of claim 6 wherein the client device is configured to:
receive data from the sensor cup; and
determine overloading, edge loading, or inadequate load distribution based on the data.

8. The dynamic hip balancing system of claim 7 wherein the client device is further configured to render a virtual representation of the plurality of sections of the sensor cup including color indicators that indicate right or wrong pressures in the plurality of sections.

9. The dynamic hip balancing system of claim 5 wherein the sensor head includes a spring coil and a piezoelectric actuator that lock the sensor head in a given position on a trial stem.

10. The dynamic hip balancing system of claim 9 further wherein the electronic connector includes an inner ball and an outer cup.

11. The dynamic hip balancing system of claim 10 wherein the sensor prosthesis is assembled by placing the sensor head in the outer cup.

12. The dynamic hip balancing system of claim 5 wherein the client device is configured to:
receive the position data from the sensor prosthesis;
receive position information from a plurality of position sensor units that are attached to bones at a plurality of body positions; and
determine correct positioning of the sensor prosthesis based on the position data and the position information from the plurality of position sensor units.

14. The dynamic hip balancing system of claim 12 wherein the client device is configured to:
receive the position data from the sensor prosthesis; and
generate a three-dimensional model of a joint including measurements based on the position data and the position information from the plurality of position sensor units, wherein the three-dimensional model assists in making rotational and longitudinal adjustments to the sensor prosthesis.

15. A dynamic shoulder balancing system comprising:
a client device; and
a tensioning device that is operated by the client device to measure a distance for shoulder prosthesis according to a given tension, the tensioning device including:
a baseplate;
a plurality of piezoelectric actuators embedded on the baseplate; and
a hemisphere that is configured above the plurality of piezoelectric actuators.

16. The dynamic shoulder balancing system of claim 15 wherein the tensioning device further include a wireless electronic module and a radio frequency identification chip that communicate position data to the client device.

17. The dynamic shoulder balancing system of claim 15 wherein the piezoelectric actuators are controllable by the client device to move the hemisphere in relation to the baseplate.

18. The dynamic shoulder balancing system of claim 15 wherein the piezoelectric actuators comprise peg structures that operate with spring coils.

19. The dynamic shoulder balancing system of claim 15 wherein the piezoelectric actuators comprise platform structures that operate with spring coils.

20. The dynamic shoulder balancing system of claim 15 wherein the piezoelectric actuators are configured to operate either independently or collectively.
